(19) United States
(12) Patent Application Publication
(10) Pub. No.: US 2005/0106209 A1
(43) Pub. Date: May 19, 2005

(54) Composition and apparatus for transdermal delivery

(76) Inventors: Mahmoud Ameri, Fremont, CA (US); Michel Cormier, Mountain View, CA (US); Yuh-Fun Maa, Millbrae, CA (US)

Correspondence Address: Francis Law Group, 1942 Embarcadero, Oakland, CA, 94606, US

(21) Appl. No.: 10/970,890

(22) Filed: Oct. 21, 2004

Related U.S. Application Data
(60) Provisional application No. 60/520,196, filed on Nov. 13, 2003.

(51) Int. Cl. A61K 31/785; A61K 31/19

(52) U.S. Cl. 424/423; 514/574; 514/566

(57) ABSTRACT

A formulation for coating a transdermal delivery device having a plurality of stratum corneum-piercing microprojections, the formulation including a biologically active agent and at least one viscosity-enhancing counterion. Preferably, the formulation has a viscosity in the range of about 20-200 cp.

### CROSS-REFERENCE TO RELATED APPLICATIONS

This application claims the benefit of U.S. Provisional Application No. 60/520,196, filed Nov. 13, 2003.

### FIELD OF THE PRESENT INVENTION

The invention relates generally to the transdermal delivery of a biologically active agent. More particularly, the invention relates a transdermal agent delivery apparatus and agent-containing formulations applied thereto.

### BACKGROUND OF THE INVENTION

The transdermal delivery of biologically active agents or drugs offers improvements over more traditional delivery methods, such as subcutaneous injections and oral delivery. Transdermal drug delivery avoids the hepatic first pass effect and gastrointestinal degradation encountered with oral drug delivery. Transdermal drug delivery also eliminates the patient discomfort, infection risk and invasiveness associated with subcutaneous injections. The term “transdermal,” as used herein, broadly encompasses the delivery of an agent or drug through a body surface, such as the skin, mucosa, or nails of an animal.

As is well known in the art, the skin functions as the primary barrier to the transdermal penetration of materials into the body. The stratum corneum, the outermost skin layer that consists of flat, dead cells filled with keratin fibers (keratinocytes) surrounded by lipid bilayers. The highly-ordered structure of the lipid bilayers confers a relatively impermeable character to the stratum corneum.

Nevertheless, transdermal delivery of therapeutic agents is an important medicament administration route. Transdermal drug delivery bypasses gastrointestinal degradation and hepatic metabolism. Most commercial transdermal drug delivery systems deliver drug by passive diffusion. The drug diffuses from a reservoir in the patch into the skin of the patient by means of the concentration gradient that exists, i.e., the drug diffuses from the high concentration in the patch reservoir to the low concentration in the patient's body. The flux of drug through a patient's skin is determined by a number of factors including the drug's partition coefficient, solubility characteristics and the permeability of the skin. Accordingly, passive diffusion delivery systems provide slow, but controlled, delivery of the drug to a patient's blood stream.

Unfortunately, many drugs exhibit transdermal diffusion fluxes that are too low to be therapeutically effective. This is especially true for high molecular weight drugs such as polypeptides and proteins. To enhance transdermal drug flux, the mechanical penetration or disruption of the outermost skin layers has been used to create pathways into the skin in order to enhance the amount of agent being transdermally delivered. Early vaccination devices known as scarifiers generally had a plurality of tines or needles which are applied to the skin to and scratch or make small cuts in the area of application. The vaccine was applied either topically on the skin, such as U.S. Pat. No. 5,487,726 issued to Rabenau or as a wetted liquid applied to the scarifier tines such as U.S. Pat. No. 4,453,926 issued to Galy, or U.S. Pat. No. 4,109,655 issued to Chacornac, or U.S. Pat. No. 3,136,314 issued to Kravitz. Scarifiers have been suggested for intradermal vaccine delivery in part because only very small amounts of the vaccine need to be delivered into the skin to be effective in immunizing the patient. Further, the amount of vaccine delivered is not particularly critical since an excess amount achieves satisfactory immunization as well as a minimum amount.

Other devices which use tiny skin piercing elements to enhance transdermal drug delivery are disclosed in European Patent EP 0407063A1, U.S. Pat. Nos. 5,879,326 issued to Godshall, et al., U.S. Pat. No. 3,814,097 issued to Ganderton, et al., U. S. Pat. No. 5,279,544 issued to Gross, et al., U. S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. These devices use piercing elements of various shapes and sizes to pierce the stratum corneum. The piercing elements disclosed in these references generally extend perpendicularly from a thin, flat member, such as a pad or sheet. The piercing elements can be extremely small, such as microprojections, having a length and width of only about 25-400 microns and a thickness of only about 5-50 microns. These microprojections make correspondingly small microslits in the stratum corneum for enhanced transdermal agent delivery therethrough.

It has further been found that applying a coating of the biologically active agent to the microprojections allows delivery of the agent into the skin. The efficiency of delivery of a biologically active agent from coated microprojections is at least partially dependent upon the area of the microprojections that extends into the skin. If the projections are long enough, the biologically active agent can be inserted into the underlying capillary bed resulting in systemic exposure to the biologically active agent. This is a desirable feature when administering drugs.

Successful transdermal drug delivery using coated microprojections requires a drug formulation having a number of characteristics. For example, the formulation must be sufficiently concentrated so that a therapeutically effective amount of drug is coated onto the microprojections to be transferred through the stratum corneum. Further, the formulation must facilitate the application of a uniform and precise coating onto the microprojections. To satisfy these requirements, an effective coating formulation must have the appropriate viscosity. Increasing the concentration of the biologically active agent also increases the viscosity. However, the concentration of the agent is usually dictated by need to provide a specific, therapeutic amount of the agent. Thus, viscosity modifiers often must be used to achieve a suitable viscosity.

Conventional viscosity modifiers include hydroxyethyl cellulose (HEC), carboxymethyl cellulose, Povidone®, Dextran® and other polymeric materials. These prior art materials present significant disadvantages when used to enhance the viscosity of protein or peptide formulations. Since the formulations are used for transdermal delivery on stratum corneum-piericing microprojections, HEC, hydroxypropyl methylcellulose (HPMC) and the like cannot be used as they are not approved excipients for parenteral applications. Other conventional viscosity enhancing agents that are approved for parenteral delivery, such as Dextran® and Povidone®, would require a substantial amount in the formulation to provide the necessary viscosity.

Due to the limited amount of interstitial fluids, materials that do not promote chemical stability of the agent (i.e., process enhancing excipients) need to be minimized to avoid compromising dissolution of the drug. Thus, the addition of significant amounts of a viscosity modifier interferes with delivery of the agent. For example, it would generally require the addition of 5-10% of Dextran® or Povidone® in a formulation to achieve suitable viscosity, an amount that would unacceptably interfere with delivery.

Accordingly, it is an object of the invention to provide a biologically active agent formulation having sufficient viscosity to facilitate a desired coating on microprojections.

It is a further object of the invention to provide a method for increasing the viscosity of a biologically active agent formulation while maintaining sufficient stability of the agent.

It is yet another object of the invention to provide a biologically active agent formulation having sufficient viscosity for efficiently coating microprojections while maintaining sufficient agent concentration to be therapeutically effective.

It is a further object of the invention to enhance the viscosity of a biologically active agent formulation for coating microprojections by adding low volatility counterions.

It is yet another object to optimize delivery of a biologically active agent coated on microprojections by enhancing the viscosity of the agent formulation.

### SUMMARY OF THE INVENTION

In accordance with the above objects and those that will be mentioned and will become apparent below, the present invention is directed to an agent-containing coating formulation for coating a transdermal delivery device having a plurality stratum corneum-piercing microprojections, the coating formulation including a biologically active agent and a viscosity-enhancing counterion, wherein the formulation has a therapeutically effective concentration of the biologically active agent. Preferably, the formulation has a viscosity in the range of about 20 cp to about 200 cp.

In a preferred embodiment, the active agent has a positive charge at the formulation pH and the viscosity-enhancing counterion comprises an acid having at least two acidic pKa. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

In other preferred embodiments, the active agent has a negative charge at the formulation pH, and the viscosity-enhancing counterion comprises a base having at least two basic pKa. Suitable bases include lysine, histidine, arginine, calcium hydroxide and magnesium hydroxide.

Another preferred embodiment is directed to a viscosity-enhancing mixture of counterions wherein the active agent has a positive charge at the formulation pH and at least one of the counterion is an acid having at least two acidic pKa. The other counterion is an acid with one or more pka. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Another preferred embodiment is directed to a viscosity-enhancing mixture of counterions, wherein the active agent has a negative charge at the formulation pH and at least one of the counterion is a base having at least two basic pKa. The other counterion is a base with one or more pka. Examples of suitable bases include sodium hydroxide, potassium hydroxide, calcium hydroxide, magnesium hydroxide, monoethanolomine, diethanolamine, triethanolamine, tromethamine, lysine, histidine, arginine, methylglucamine, glucosamine, ammonia, and morpholine.

Generally, in the noted embodiments of the invention, the amount of counterion should neutralize the charge of the biologically active agent.

The counterion or the mixture of counterions is present in amounts necessary to neutralize the charge present on the agent at the pH of the formulation. Excess of counterion (as the free acid or as a salt) can be added to the peptide in order to control pH and to provide adequate buffering capacity.

In one embodiment of the invention, the biologically active agent is selected from the group consisting of ACTH (1-24), calcitonin, desmopressin, LHRH, goserelin, leuprolide, buserelin, triptorelin, other LHRH analogs, PTH, PTH (1-34), vasopressin, deamino [val4, D-Arg8] arginine vasopressin, interferon alpha, interferon beta, interferon gamma, FSH, EPO, GM-CSF, G-CSF, IL-10, glucagon, GRF, analogs thereof and pharmaceutically acceptable salts thereof.

In one preferred embodiment, the agent comprises PTH (1-34) and the counterion is a viscosity-enhancing mixture of counterions chosen from the group of citric acid, tartaric acid, malic acid, hydrochloric acid, glycolic acid, and acetic acid.

The invention is further directed to a transdermal delivery device having a microprojection member that includes a plurality of microprojections that are adapted to pierce through the stratum corneum into the underlying epidermis and dermis layers of the skin, the microprojection member further including a biologically active agent, wherein the coating is formed from a formulation having at least one viscosity-enhancing counterion.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one embodiment of a microprojection array that is suitable for practice of the invention;

FIG. 2 is a perspective view of the microprojection array shown in FIG. 1 with a coating deposited on the microprojections;

FIG. 3 is a graph showing the oxidation of various compositions of the invention as a function of time;

FIG. 4 is a graph showing the purity of various compositions of the invention as a function of time; and

FIG. 5 is a graph showing the aggregation of various compositions of the invention as a function of time.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “biologically active agent”, as used herein, refers to a composition of matter or mixture containing a drug which is pharmacologically effective when administered in a therapeutically effective amount. Presently preferred agents of the invention comprise peptides and proteins. Examples of such active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10) and glucagon. It is to be understood that more than one agent may be incorporated into the agent formnulation in the method of this invention, and that the use of the term “active agent” in no way excludes the use of two or more such agents or drugs.

The term “biologically active agent”, as used herein, also refers to a composition of matter or mixture containing a vaccine or other immunologically active agent or an agent which is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, and diphtheria vaccine, recombinant protein vaccines, DNA vaccines and therapeutic cancer vaccines. The term “vaccine” thus includes, without limitation, antigens in the form of proteins, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of agent employed in the coatings will be that amount necessary to deliver a therapeutically effective amount of the agent to achieve the desired therapeutic result.

In practice, this will vary widely depending upon the particular biologically active agent being delivered, the site of delivery, the severity of the condition being treated, the desired therapeutic effect and the dissolution and release kinetics for delivery of the agent from the coating into skin tissues. It is not practical to define a precise range for the therapeutically effective amount of the biologically active agent incorporated into the microprojections and delivered transdermally according to the methods described herein.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

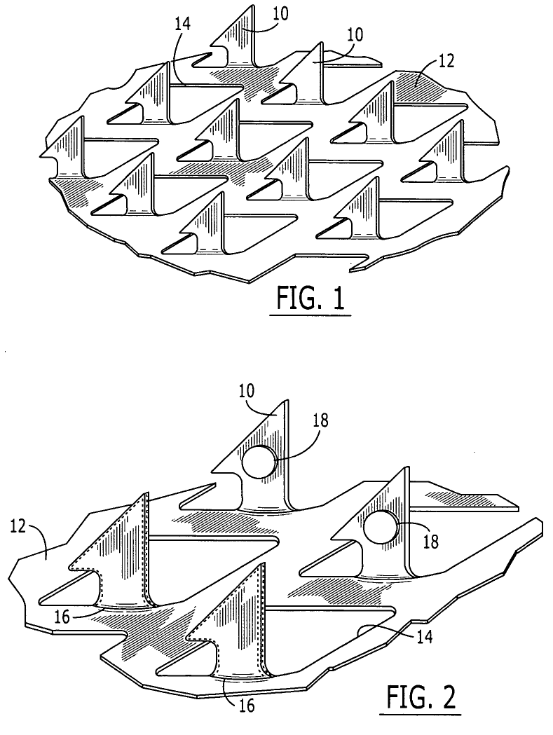

The term “microprojection array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration such as that shown in FIG. 1. The microprojection array may also be formed in other known manners, such as by forming one or more strips having microprojections along an edge of each of the strip(s) as disclosed in Zuck, U.S. Pat. No. 6,050,988. The microprojection array may include hollow needles which hold a dry pharmacologically active agent.

References to the area of the sheet or member and reference to some property per area of the sheet or member are referring to the area bounded by the outer circumference or border of the sheet.

The term “solution” or “formulation” shall include not only compositions of fully dissolved components but also suspensions of components including, but not limited to, protein virus particles, inactive viruses, and split-virions.

The term “pattern coating”, as used herein, refers to coating an agent onto selected areas of the microprojections. More than one agent may be pattern coated onto a single microprojection array. Pattern coatings can be applied to the microprojections using known micro-fluid dispensing techniques such as micropipeting and ink jet coating.

As indicated above, the present invention provides a formulation of a biologically active agent to a patient in need thereof, wherein the formulation has enhanced viscosity to facilitate coating on a plurality of stratum corneum-piercing microprojections.

According to the invention, the viscosity of a biologically active agent formulation is enhanced by addition of counterions. Preferably, the agent comprises a peptide or protein. The interaction of the peptide or protein with the counterions leads to an increase in viscosity due to the formation of secondary bonds or hydrogen bonds. The counterions employed require only small quantities to have a marked increase on the viscosity of the formulation. For coatability, using the dip-coating methods described above, a formulation has to be within a certain viscosity range. A presently preferred viscosity is in the range of about 20-200 centipoise (cp). Using a formulation that has an unacceptable viscosity, for example, less than about 20 cp or greater than about 200 cp results in high coating variability.

In a preferred embodiment, the agent has a positive charge at the formulation pH and wherein the viscosity-enhancing counterion comprises an acid having at least two acidic pKa. Suitable acids include, but not limited to, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

In other preferred embodiments, the agent has a negative charge at the formulation pH, and the viscosity-enhancing counterion comprises a base having at least two basic pKa. Suitable bases include, but are not limited to, lysine, histidine, arginine, calcium hydroxide and magnesium hydroxide.

Another preferred embodiment is directed to a viscosity-enhancing mixture of counterions wherein the agent has a positive charge at the formulation pH and at least a first counterion is an acid having at least two acidic pKa. A second counterion is an acid with one or more pka. Examples of suitable acids include, but not limited to, hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Another preferred embodiment is directed to a viscosity-enhancing mixture of counterions wherein the agent has a negative charge at the formulation pH and a first counterion is a base having at least two basic pKa. A second counterion is a base with one or more pka. Examples of suitable bases include, but are not limited to, sodium hydroxide, potassium hydroxide, calcium hydroxide, magnesium hydroxide, monoethanolomine, diethanolamine, triethanolamine, tromethamine, lysine, histidine, arginine, methylglucamine, glucosamine, ammonia, and morpholine.

Generally, in the noted embodiments of the invention, the amount of counterion (or mixture of counterions) should neutralize the net charge of the biologically active agent.

The counterion or the mixture of counterions is present in amounts necessary to neutralize the net charge present on the agent at the pH of the formulation. Excess of counterion (as the free acid or as a salt) can be added to the peptide in order to control pH and to provide adequate buffering capacity.

Preferably, the ratio of net charges between the counterion or the mixture of counterions to the biologically active agent is 1-20 (e.g., for every net charge present on the biological active agent, there is at least 1 and up to 20 net charges of counterion or mixture of counterions). More preferably the ratio of net charges between the counterion (or mixture of counterions) to the biologically active agent is 1-10. Even more preferably, the ratio of net charges between the counterion (or mixture of counterions) to the biologically active agent is 1-5.

In one embodiment of the invention, the biologically active agent is selected from the group comprising of ACTH (1-24), calcitonin, desmopressin, LHRH, goserelin, leuprolide, buserelin, triptorelin, other LHRH analogs, PTH, PTH (1-34), vasopressin, deamino [val4, D-Arg8] arginine vasopressin, interferon alpha, interferon beta, interferon gamma, FSH, EPO, GM-CSF, G-CSF, IL-10, glucagon, GRF, analogs thereof and pharmaceutically acceptable salts thereof.

In a preferred embodiment, the agent comprises PTH (1-34) and the counterion is a viscosity-enhancing mixture of counterions chosen from the group comprising citric acid, tartaric acid, malic acid, hydrochloric acid, glycolic acid and acetic acid.

The invention also comprises a method for applying a coating of a biologically active agent to a transdermal delivery device having a plurality of stratum corneum-piercing microprojections, comprising the steps of providing a formulation of the biologically active agent, enhancing the viscosity of the formulation by adding counterions while maintaining a therapeutically effective concentration of the biologically active agent, and applying the formulation to the microprojections. Preferably, counterions are added to the formulation to achieve a viscosity in the range of about 20-200 cp.

Preferably, the methods of the invention produce a coating thickness of less than about 10 microns.

According to the invention, the agent formulation is used to apply a preferably uniform coating to a microprojection transdermal delivery device. The microprojections are adapted to pierce through the stratum corneum into the underlying epidermis layer, or epidermis and dermis layers. The applied formulation is dried onto the microprojections to form a dry coating thereon which contains the biologically active agent. Upon piercing the stratum corneum layer of the skin, the agent-containing coating is dissolved by body fluid (intracellular fluids and extracellular fluids, such as interstitial fluid) and released into the skin for local or systemic therapy.

The kinetics of the agent-containing coating dissolution and release will depend on many factors including the nature of the biologically active agent, the coating process, the coating thickness and the coating composition (e.g., the presence of coating formulation additives). Depending on the release kinetics profile, it may be necessary to maintain the coated microprojections in piercing relation with the skin for extended periods of time (e.g., up to about 8 hours). This can be accomplished by anchoring the microprojection member to the skin using adhesives or by using anchored microprojections such as described in WO 97/48440, incorporated by reference in its entirety.

FIG. 1 illustrates one embodiment of a stratum corneum-piercing microprojection member for use with the present invention. FIG. 1 shows a portion of the member having a plurality of microprojections 10. The microprojections 10 extend at substantially a 90° angle from sheet 12 having openings 14. Sheet 12 may be incorporated into a delivery patch, including a backing for sheet 12, and may additionally include adhesive for adhering the patch to the skin. In this embodiment, the microprojections are formed by etching or punching a plurality of microprojections 10 from a thin metal sheet 12 and bending microprojections 10 out of the plane of the sheet.

Metals, such as stainless steel and titanium, are the preferred materials for constructing the illustrated patch. Metal microprojection members are disclosed in Trautman, et al., U.S. Pat. No. 6,083,196; Zuck, U.S. Pat. No. 6,050,988; and Daddona, et al., U.S. Pat. No. 6,091,975; the disclosures of which are incorporated herein by reference.

Other microprojection members that can be used with the present invention are formed by etching silicon using silicon chip etching techniques or by molding plastic using etched micro-molds. Silicon and plastic microprojection members are disclosed in Godshall, et al., U.S. Pat. No. 5,879,326, the disclosures of which is incorporated herein by reference.

FIG. 2 illustrates the microprojection member having microprojections 10 with a coating 16 that preferably contains at least one biologically active agent and optionally, a vasoconstrictor. The coating 16 may partially or completely cover the microprojection 10. For example, the coating can be in a dry pattern coating 18 on the microprojections. The coatings can be applied before or after the microprojections are formed.

According to the invention, the inventive formulations of the invention can be coated on the microprojections 10 by a variety of known methods. One such method is dip-coating. Dip-coating can be described as a means to coat the microprojections by partially or totally immersing the microprojections into the coating solution. Alternatively, the entire device can be immersed into the coating solution. Preferably, only those portions of the microprojection member that pierce the skin are coated.

By use of the partial immersion technique described above, it is possible to limit the coating to only the tips of the microprojections. There is also a roller coating mechanism that limits the coating to the tips of the microprojection. This technique is described in U.S. Provisional Application No. 60/276,762, filed 16 Mar. 2001, which is fully incorporated herein by reference.

Other coating methods include spraying the coating solution onto the microprojections. Spraying can encompass formation of an aerosol suspension of the coating composition. In a preferred embodiment an aerosol suspension having a droplet size of about 10 to 200 picoliters is sprayed onto the microprojections and then dried.

In another embodiment, a very small quantity of the coating solution can be deposited onto the microprojections 10, as shown in FIG. 2 as pattern coating 18. The pattern coating 18 can be applied using a dispensing system for positioning the deposited liquid onto the microprojection surface. The quantity of the deposited liquid is preferably in the range of 0.5 to 20 nanoliters/microprojection. Examples of suitable precision-metered liquid dispensers are disclosed in U.S. Pat. Nos. 5,916,524; 5,743,960; 5,741,554; and 5,738,728; the disclosures of which are fully incorporated herein by reference.

Microprojection coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

The desired coating thickness is dependent upon the density of the microprojections per unit area of the sheet and the viscosity and concentration of the coating composition as well as the coating method chosen. Preferably, the coating thickness should be less than 50 microns, more preferably, less than 25 microns, since thicker coatings have a tendency to slough off the microprojections upon stratum corneum piercing. Generally coating thickness is referred to as an average coating thickness measured over the coated microprojection.

As indicated, in one embodiment, the coating thickness is preferably less than 10 microns, as measured from the microprojection surface. More preferably, the coating thickness is in the range of approximately 1 to 10 microns.

The active agent used in the present invention requires that the total amount of agent coated on all of the microprojections of a microprojection array be in the range of 1 microgram to 1 milligram.

Amounts within this range can be coated onto a microprojection array of the type shown in FIG. 1 having the sheet 12 with an area of up to 10 cm² and a microprojection density of up to 1000 microprojections per cm².

As indicated above, the coatings of the invention comprise at least one biologically active agent and at least one viscosity-enhancing counterion. It has been found that addition of the counterion increases the viscosity of the agent formulation, improving the consistency of the coating on a microprojection transdermal delivery device.

Also preferably, microprojection array 10 is reproducibly and uniformly applied to a patient through the use of an applicator, for example a biased (e.g., spring driven) impact applicator. Such devices are described in Trautman et al., U.S. patent application Ser. No. 09/976,673, filed Oct. 12, 2001, the disclosure of which is incorporated herein by reference. Most preferably, the coated microprojection array is applied with an impact of at least 0.05 joules per cm² of the microprojection array in 10 msec or less.

### EXAMPLES

The following examples are provided to enable those skilled in the art to more clearly understand and practice the present invention. They should not be considered as limiting the scope of the invention, but merely as being illustrated as representative thereof.

The examples demonstrate the utilization of a weak acid with a peptide or protein agent to enhance the viscosity. The interaction of the weak acid anion with the positively charged peptide or protein apparently leads to the formation of secondary bonds, e.g. hydrogen bonds, which results in an increase in solution viscosity. The greater the number of acidic groups, the greater the number of secondary bonds formed between the anions and the peptide or protein, hence the greater the viscosity increase. Thus, the theoretical viscosity enhancing capabilities increase when monoacids, di-acids, tri-acids and tetra-acids are compared.

Parathyroid Hormone (PTH) is an eighty-four amino acid polypeptide that regulates calcium homeostasis in serum by stimulation of calcium resorption in the kidney by enhancing resorption of calcified bone matrix. In addition it also stimulates bone forming processes. It is the first (N-terminal) thirty-four amino acids that are responsible for the hormonal activity. Consequently, a synthetic preparation of the first thirty-four amino acids, PTH (1-34), was evaluated.

Various weak acid buffers have been incorporated in some PTH (1-34) formulations in these experiments. A control formulation included PTH (1-34) actate with sucrose was also prepared. The experiments investigate the physicochemical properties afforded to PTH (1-34) by various mixtures of mono-, di- and tri- acids and the stability of the solution formulations over a 48 hr period at 2-8° C. The PTH (1-34) formulations were buffered to a pH 5.2.

Table 1 provides the lot numbers and manufacturers of the raw materials utilized. Table 2 provides the eight formulations manufactured for the solution stability study. The formulations were prepared by dispensing 20 mg of PTH (1-34) into a 1.5 ml polypropylene eppendorf centrifuge tube. Another 1.5 ml polypropylene eppendorf centrifuge tube was charged the appropriate amount of sterile water, buffer (if required for formulation), sucrose (if required for formulation) and polysorbate 20 solution. The centrifuge vial containing the excipients was allowed to dissolve and was centrifuged for a period of 1 minute at 7000 rpm utilizing a Fisher Scientific mini centrifuge, model MicroV. The excipient solution was dispensed into the centrifuge vial containing the PTH(1-34) which was subsequently placed in a rotator, Glas-Col, model No. 099A RD4512. Dissolution of the PTH (1-34) with the excipient solution was conducted at 2-8° C.

The PTH (1-34) solution formulation was centrifuged for a period of2 minutes at 7000 rpm utilizing a Fisher Scientific mini centrifuge, model MicroV. Viscosity of the solution formulations were conducted utilizing a Brookfield viscometer, model CAP2000. All viscosity measurements were conducted utilizing cone and plate geometry, with a cone angle of 0.45° and radius 1.511 cm. Shear rate was set to 2667 s⁻¹ and temperature was maintained at 10° C. during viscosity measurement. Viscosities were calculated by the CAPCALC™ software. The viscosity measurements utilized 70 μl of PTH (1-34) solution formulation.

Decomposition of PTH via oxidation in all formulations was measured by a stability-indicating reverse phase high pressure liquid chromatography (RP-HPLC) (UV detection at 215 nm). Oxidized PTH was separated from native PTH using a Zorbax 300 SB-C8 reversed phase column (4.6 mm ID×150 mm, 3.51 μm) (Agilent Technologies, Inc. CA, USA) maintained at 55° C. Final chromatographic conditions involved a gradient elution, with solvent A: 0.1% trifluoroacetic acid in water, and solvent B: 0.09% trifluoroacetic acid in acetonitrile. The pump flow rate was 1 mL/min. Soluble aggregates (covalent dimer and higher order) were determined by size exclusion high pressure liquid chromatography (HPLC) (UV detection at 214 nm) using a TCK-gel G2000 SWXL column (7.8 mm ID×300 mm, 5 μm) (Toso Haas, Japan) with an isocratic mobile phase consisting of 0.1% trifluoroacetic acid in 0.2M NaCl and acetonitrile (70/30 by volume), at a flow rate of 0.5 mL/min. Chromatography for both assays was performed with Agilent 1100 series HPLC systems (Agilent Technologies, Inc., CA, USA) provided with a binary pump, a thermostatted autosampler, a thermostatted column compartment and a multiple wavelength DAD/UV detector. Data was collected and analyzed using a Turbochrom Client Server Software, version 6.2 (Perkin Elmer, Inc).

TABLE 1

| Material | Lot No. | Manufacturer |
|---|---|---|
| PTH (1-34) acetate | FPTH9801D | BACHEM |
| Sucrose | 27412A | Pfanstiehl |
| Tartaric acid (L(+)) | 27H0743 | Sigma |
| Citric acid | 126H0743 | Sigma |
| Malic acid (DL) | EF02109PT | Sigma |
| Glycolic acid | 106F7703 | Sigma |
| HCl | 1202157 | Ricca |
| Polysorbate 20 | MV0208184 | Croda |
| Water for injection | 79-306-DK | Abbot Laboratories |

TABLE 2

| Formulation | Formulation Composition | Formulation |
|---|---|---|
| ID | (% w/w) | Lot No. |
| A | 20% PTH, 0.2% Tween 20 | 7528070C |
| B | 20% PTH, 0.5% HCl, 0.2% Tween 20 | 7528070D |
| C | 20% PTH, 20% Sucrose, 0.2% Tween 20 | 7528069A |
| D | 20% PTH, 20% Sucrose, 0.5% HCl, | 7528069B |
| | 0.2% Tween 20 | |
| E | 20% PTH, 20% Sucrose, 1.2% glycolic | 7528069C |
| | acid, 0.2% Tween 20 | |
| F | 20% PTH, 20% Sucrose, 1.4% malic acid, | 7528069D |
| | 0.2% Tween 20 | |
| G | 20% PTH, 20% Sucrose, 1.2% tartaric acid, | 7528070A |
| | 0.2% Tween 20 | |
| H | 20% PTH, 20% Sucrose, 1.7% citric acid, | 7528070B |
| | 0.2% Tween 20 | |

Viscosity results of the formulations are shown in Table 3. Citric and malic acid buffered formulations exhibited the largest increase viscosity enhancement compared to the control formulation (Lot No. 7528069A). It is interesting to note that citric acid, a tri-acid, yielded a formulation with the highest viscosity. Based on the results given in Table 3, the trend for viscosity enhancement following addition of weak acid buffers is tri-acid to di-acid to mono-acid.

TABLE 3

| Formulation Lot No. | Viscosity (cP) |
|---|---|
| 7528069A | 68 |
| 7528069B | 87 |
| 7528069C | 53 |
| 7528069D | 116 |
| 7528070A | 77 |
| 7528070B | 172 |

Presumably, viscosity enhancement of the weak acid buffers is achieved by the interaction of the weak acid anion with the positively charged PTH. This leads to the formation of secondary bonds, e.g. H-bonds, which results in an increase in solution viscosity. The greater the number of acidic groups the greater the number of secondary bonds formed between the anions and the PTH, hence, the greater the viscosity increase.

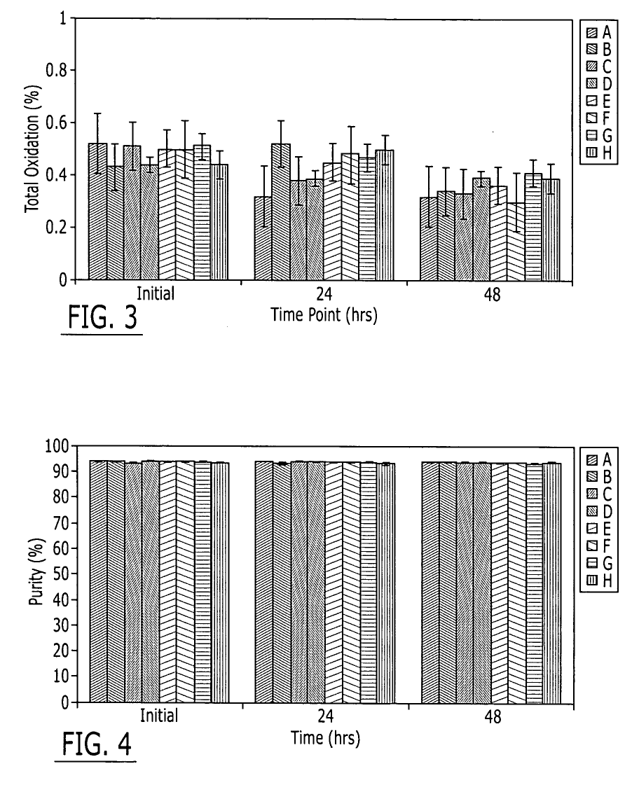

The overall stability of the PTH formulations was determined and the results are shown in FIGS. 3-5. Total oxidized PTH (1-34) and purity of the formulations were determined by RPHPLC the results are shown in FIGS. 3 and 4, respectively.

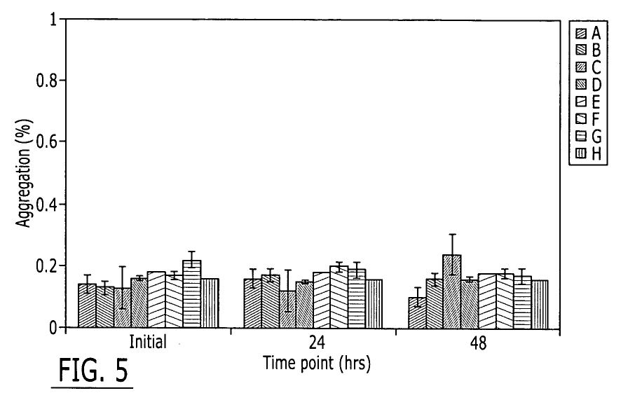

From FIG. 3 it is apparent, within the variability of the results, that the total oxidized product does not increase markedly over the 48 hour period, similarly the purity shown in FIG. 4 of the PTH (1-34) solution formulations remained constant during the course of the study. SEC was utilized to measure the propensity of the PTH (1-34) solution formulations for aggregation and formation of covalent high molar mass products. The results are summarized in FIG. 5, which shows formulations of PTH (1-34) did not aggregate appreciably over the 48 hour period when stored at 2-8° C.

The data above demonstrates that counterion mixtures of citric acid/acetic acid, malic acid/acetic acid, tartaric acid/ acetic acid and hydrochloric acid/acetic acid increase the viscosity of hPTH (1-34) with respect to the control formulation. Total oxidized PTH (1-34) product, purity and aggregation remained uniform for all formulations during the course of the study.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A composition for coating a transdermal delivery device having stratum corneum-piercing microprojections comprising a formulation of a biologically active agent and a viscosity-enhancing counterion, wherein said formulation has a therapeutically effective concentration of said biologically active agent.

2. The composition of claim 1, wherein said formulation has a viscosity in the range of about 20 cp to about 200 cp.

3. The composition of claim 1, wherein said formulation has a first pH value, wherein said biologically active agent has a positive charge at said formulation pH, and wherein said viscosity-enhancing counterion comprises a first acid.

4. The composition of claim 3, wherein said first acid has at least two acidic pKa values.

5. The composition of claim 4, wherein said first acid is selected from the group consisting of maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, carbonic acid, sulfuric acid, and phosphoric acid.

6. The composition of claim 3, wherein said viscosity-enhancing counterion further includes a second acid.

7. The composition of claim 6, wherein said second acid has at least one acidic pKa value.

8. The composition of claim 7, wherein said second acid is selected from the group consisting of hydrochloric acid, hydrobromic acid, nitric acid, sulfonic acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, tricarballylic acid and ethylenediaminetetraacetic acid.

9. The composition of claim 1, wherein said formulation has a second pH value, wherein said biologically active agent has a negative charge at said formulation second pH value, and wherein said viscosity-enhancing counterion comprises a first base.

10. The composition of claim 9, wherein said first base has at least two basic pKa values.

11. The composition of claim 10, wherein said first base is selected from the group consisting of lysine, histidine, arginine, calcium hydroxide and magnesium hydroxide.

12. The composition of claim 9, wherein said viscosity-enhancing counterion further includes a second base.

13. The composition of claim 12, wherein said second base has at least one basic pKa value.

14. The composition of claim 13, wherein said second base is selected from the group consisting of sodium hydroxide, potassium hydroxide, calcium hydroxide, magnesium hydroxide, monoethanolomine, diethanolamine, triethanolamine, tromethamine, lysine, histidine, arginine, methylglucamine, glucosamine, ammonia, and morpholine.

15. The composition of claim 1, comprising an amount of said viscosity-enhancing counterion sufficient to neutralize a charge of said biologically active agent.

16. The composition of claim 1, wherein said biologically active agent is selected from the group consisting of ACTH (1-24), calcitonin, desmopressin, LHRH, goserelin, leuprolide, buserelin, triptorelin, other LHRH analogs, PTH, PTH (1-34), vasopressin, deamino [val4, D-Arg8] arginine vasopressin, interferon alpha, interferon beta, interferon gamma, FSH, EPO, GM-CSF, G-CSF, IL-10, glucagon, GRF, analogs thereof and pharmaceutically acceptable salts thereof.

17. The composition of claim 16, wherein said viscosity-enhancing counterion comprises one or more acids selected from the group consisting of citric acid, tartaric acid, malic acid, hydrochloric acid, glycolic acid, and acetic acid.

18. The composition of claim 17, wherein said biologically active agent comprises PTH (1-34).

19. An apparatus for transdermally delivering a biologically active agent to a subject, comprising a microprojection member having a plurality of microprojections that are adapted to pierce said subjects stratum corneum, said microprojection member including a biocompatible coating having at least one biologically active agent, wherein said coating is formed from a formulation having at least one viscosity-enhancing counterion.

20. The apparatus of claim 19, wherein said formulation has a viscosity in the range of about of about 20-200 cp.

21. The apparatus of claim 19, wherein said biocompatible coating has a coating thickness less than about 10 microns.

22. The apparatus of claim 19, wherein said formulation has a first pH value and said biologically active agent has a positive charge at said formulation first value.

23. The apparatus of claim 22, wherein said formulation includes a first viscosity-enhancing counterion having at least two acidic pKa values.

24. The apparatus of claim 23, wherein said formulation includes a second viscosity-enhancing counterion, said second viscosity-enhancing counterion having at least one acidic pKa value.

25. The apparatus of claim 19, wherein said formulation has a second pH value and said biologically active agent has a negative charge at said formulation second pH value.

26. The apparatus of claim 25, wherein said formulation includes a first viscosity-enhancing counterion having at least two basic pKa values.

27. The apparatus of claim 26, wherein said formulation includes a second viscosity-enhancing counterion, said second viscosity-enhancing counterion having at least one basic pKa value.

28. The apparatus of claim 23, wherein said first viscosity-enhancing counterion has sufficient activity to neutralize a charge of said biologically active agent.
